(19) United States
(12) Patent Application Publication
(10) Pub. No.: US 2025/0195276 A1
(43) Pub. Date: Jun. 19, 2025

(54) HEAD-MOUNTED EARMUFF DEVICE

(71) Applicant: Jing-Feng WU, Tainan (TW)

(72) Inventors: Jing-Feng WU, Tainan (TW); Yi-Kai SUNG, Tainan (TW)

(21) Appl. No.: 18/441,582

(22) Filed: Feb. 14, 2024

(30) Foreign Application Priority Data
Dec. 13, 2023 (TW) 112148447

(51) Int. Cl. A61F 11/14

(52) U.S. Cl. CPC A61F 11/14

(57) ABSTRACT

The invention discloses a head-mounted earmuff device comprising: two earmuff bodies and a headband body. Each earmuff body has an earpad portion and a housing portion. The earpad portion has an accommodating hole. The housing portion is pivotably connected to the earpad portion. The housing portion covers the outer side surface of the earpad portion to isolate environmental sounds. The housing portion is pivoted to leave away from the outer side surface of the earpad portion to allow reception of the environmental sounds. The headband body has a mounting member and two assembling portions, and a pivoting end of the assembling portion is pivotably connected to the earpad portion. A sliding end of the assembling portion is slidably connected to a mounting section of the mounting member, and a dimension of a head-mounted space is adjustable by sliding and fixing the two assembling portions with respect to the mounting member.

### FIELD OF THE INVENTION

The present invention relates to earmuffs, and more particularly relates to a head-mounted earmuff device.

### BACKGROUND OF THE INVENTION

An earmuff is a hearing protection device which is worn on a user's head and covers a user's ears to protect the user's ears from noise to prevent from noise-induced hearing loss.

However, when there is a need to have a conversation or use a telecommunications device, the user often unavoidably needs to take off the whole earmuffs, causing the user to be exposed to noise. Alternatively, the user may lift up and bend the earmuff on one side to expose only one ear to hear the conversation or hear on the telecommunications device, but this may cause the earmuffs to press strongly against the user's head, resulting in discomfort.

### SUMMARY OF THE INVENTION

Therefore, one objective of the present invention is to provide a head-mounted earmuff device which has earmuff bodies switchable to an opened position to allow reception of the environmental sounds without taking off the earmuffs. The head-mounted earmuff device also has an advantage that the user could wear the head-mounted earmuff device to hear outside without the necessity of lifting up and bending the earmuff on one side.

In order to overcome the technical problems in the prior art, the present invention provides a head-mounted earmuff device comprising: two earmuff bodies, each earmuff body having an earpad portion and a housing portion, wherein an inner side surface of the earpad portion serves as a wearing surface which abuts a user's head, the earpad portion is provided with an accommodating hole formed through the earpad portion from the inner side surface of the earpad portion to an outer side surface of the earpad portion, and the housing portion is pivotably connected to the earpad portion in a manner that the housing portion is pivoted with respect to the earpad portion to switch between a closed position and an opened position, wherein in the closed position, the housing portion covers the outer side surface of the earpad portion so that the accommodating hole is closed to isolate environmental sounds, and in the opened position, the housing portion is pivoted to leave away from the outer side surface of the earpad portion so that the accommodating hole is opened to allow reception of the environmental sounds; and a headband body having a mounting member and two assembling portions, wherein the mounting member has two mounting sections respectively extending in an arc toward left side and right side of the mounting member, the assembling portion has a pivoting end extending downward and a sliding end extending upward, the pivoting end of the assembling portion is pivotably connected to the earpad portion of the earmuff body, the sliding end of the assembling portion is slidably connected to the mounting section in an adjustable and fixable connecting length between the assembling portion and the mounting member, and a dimension of a head-mounted space defined by the mounting member and the two assembling portions is adjustable by sliding and fixing the two assembling portions with respect to the mounting member.

In one embodiment of the present invention, the head-mounted earmuff device is provided, wherein the earpad portion of the earmuff body includes an annular frame and a cushion body mounted to an inner side surface of the annular frame.

In one embodiment of the present invention, the head-mounted earmuff device is provided, wherein the annular frame is provided with two pivoting studs respectively protruding from a front side and a back side of the annular frame so that the pivoting end of the assembling portion is pivotably connected to the two pivoting studs protruding from the front side and the back side of the annular frame, and the pivoting stud is provided with a pivot-stop block outward protruding from the pivoting stud, the assembling portion is provided at an inner side surface of the pivoting end with an inner-restriction stop and an outer-restriction stop which are spaced apart from each other according to a pivot-restriction angle, the pivot-stop block is provided between the inner-restriction stop and the outer-restriction stop to confine the earmuff body to pivot with respect to the assembling portion in a pivot-angle range.

In one embodiment of the present invention, the head-mounted earmuff device is provided, wherein the housing portion of the earmuff body has an outer case and a soundproof block, the outer case is pivotably connected to the annular frame, the soundproof block is disposed in the outer case, and the soundproof block is arranged to abut an outer side surface of the annular frame in the closed position to close the accommodating hole and leave away from the annular frame in the opened position to open the accommodating hole.

In one embodiment of the present invention, the head-mounted earmuff device is provided, wherein the annular frame is provided with a pivoting base located in a top part of the annular frame, the outer case is provided with a pivoting portion located in a top part of the outer case, the outer case is pivotably connected to the annular frame by connecting the pivoting portion to the pivoting base, the outer case is flipped to the opened position, taking the pivoting base as a pivot center, from bottom to top with respect to the annular frame by a side pulling force applied to the outer case to leave away from the annular frame, and the outer case is pivoted from the opening position to the closing position by gravity when the side pulling force is removed.

In one embodiment of the present invention, the head-mounted earmuff device is provided, wherein the annular frame is provided with a magnetic member, the outer case is provided with a corresponding magnetic element corresponding to the magnetic member so that the housing portion and the earpad portion are fixed in the closed position by a mutual magnetic attraction between the magnetic member and the corresponding magnetic element.

In one embodiment of the present invention, the head-mounted earmuff device is provided, wherein the annular frame is provided with an outer-seal flange protruding outward from the annular frame, the outer case is provided with an inner-seal flange protruding inward from the outer case, an outer-annular contour of the inner-seal flange is corresponding in shape to an inner-annular contour of the outer-seal flange, wherein in the closed position, the inner-seal flange of the outer case is air-tightly connected to the outer-seal flange of the annular frame to prevent the environmental sounds from entering through an aperture between the annular frame and the outer case.

In one embodiment of the present invention, the head-mounted earmuff device is provided, wherein a slide-fixing device is provided between the mounting section of the mounting member and the sliding end of the assembling portion, the slide-fixing device includes a fixing rack extending in the mounting section along the mounting member and a fixing detent pivotably connected to the sliding end of the assembling portion in an elastically restorable manner, wherein the fixing detent has a fixing-protrusion part which is detachably engaged with the fixing rack, a torsion spring is disposed between the fixing detent and the mounting member to depress against the fixing detent to maintain the fixing-protrusion part of the fixing detent to be engaged with the fixing rack, and when an external pressing force is applied to the fixing detent to overcome a biasing force of the torsion spring, the fixing detent is upward pivoted so that the fixing-protrusion part leaves away from the fixing rack, and thereby the assembling portion is allowed to be slidable with respect to the mounting member.

In one embodiment of the present invention, the head-mounted earmuff device is provided, wherein the sliding end of the assembling portion is provided with an accommodating recess recessed inward from the sliding end of the assembling portion along the assembling portion to form an arc accommodating space, the mounting section of the mounting member is mounted into the accommodating recess along the assembling portion, a guide-protrusion part protrudes from an arc inner side surface of the mounting member along the mounting member, a guiding groove is recessed inside the assembling portion along the assembling portion, the guide-protrusion part is mounted to the guiding groove to guide a relative displacement between the mounting member and the assembling portion.

In one embodiment of the present invention, the head-mounted earmuff device is provided, wherein the mounting member is provided with a stopping block bent toward the mounting member at an end part of the mounting section, the assembling portion is provided with a resisting block located inside the assembling portion, when the mounting member is outward extracted with respect to the assembling portion, the stopping block knocks against the resisting block, and thereby the mounting member and the assembling portion are prevented from detaching each other.

The head-mounted earmuff device of the present invention has the technical effects as follows. The housing portion can cover the outer side surface of the earpad portion to close the accommodating hole to isolate environmental sounds, and the housing portion can be pivoted to leave away from the outer side surface of the earpad portion to open the accommodating hole to allow reception of the environmental sounds. The dimension of the head-mounted space defined by the mounting member and the two assembling portions is adjustable by sliding and fixing the two assembling portions with respect to the mounting member.

### BRIEF DESCRIPTION OF THE DRAWINGS

FIG. 1 is a schematic perspective view illustrating a head-mounted earmuff device according to one embodiment of the present invention;

FIG. 2 is a schematic sectional view illustrating the head-mounted earmuff device according to the embodiment of the present invention;

FIG. 3 is a schematic drawing illustrating the head-mounted earmuff device according to the embodiment of the present invention when a housing portion of a earmuff body is in an opened position;

FIG. 4 is a schematic partial enlarged view illustrating an annular frame and a pivoting end of the head-mounted earmuff device according to the embodiment of the present invention;

FIG. 5 is a schematic partial sectional view illustrating a mounting member and an assembling portion of the head-mounted earmuff device according to the embodiment of the present invention; and

FIG. 6 is another schematic partial sectional view illustrating the mounting member and the assembling portion of the head-mounted earmuff device according to the embodiment of the present invention.

### DETAILED DESCRIPTION OF THE PREFERRED EMBODIMENTS

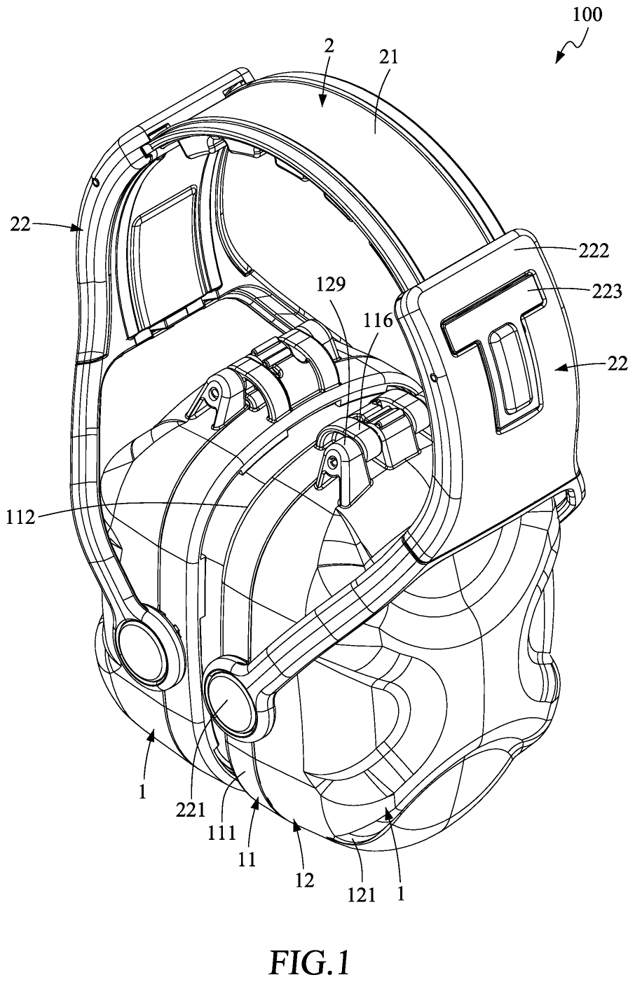

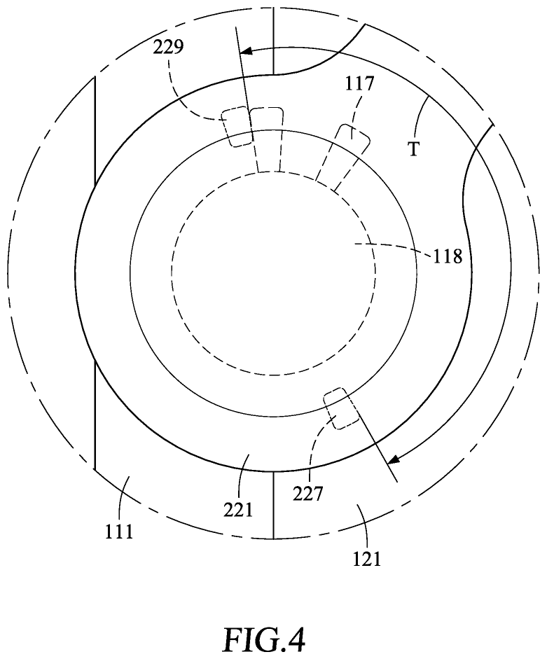

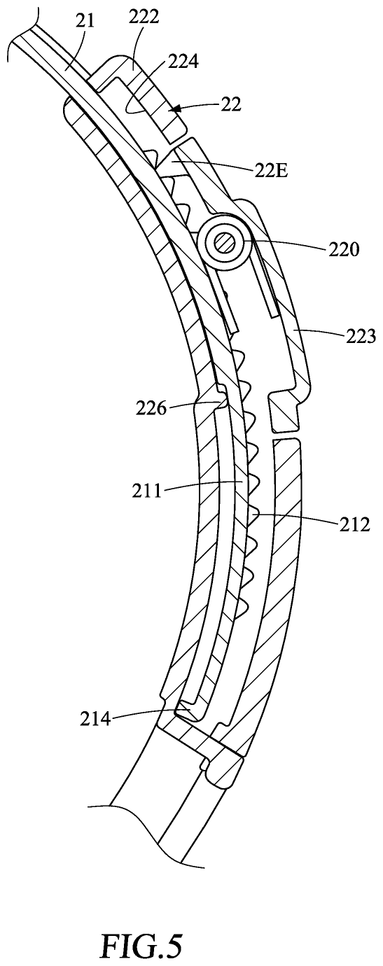

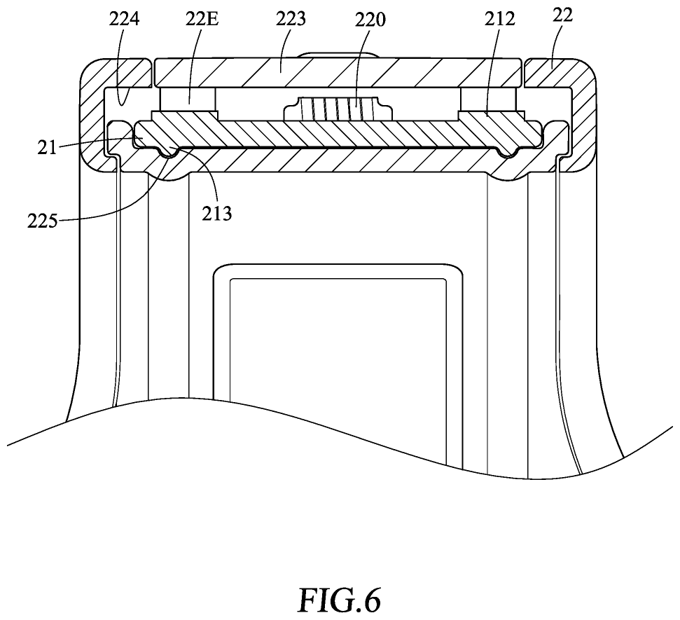

The preferred embodiments of the present invention are described in detail with reference to FIGS. 1 to 6. The description is used for explaining the embodiments of the present invention only, but not for limiting the scope of the claims.

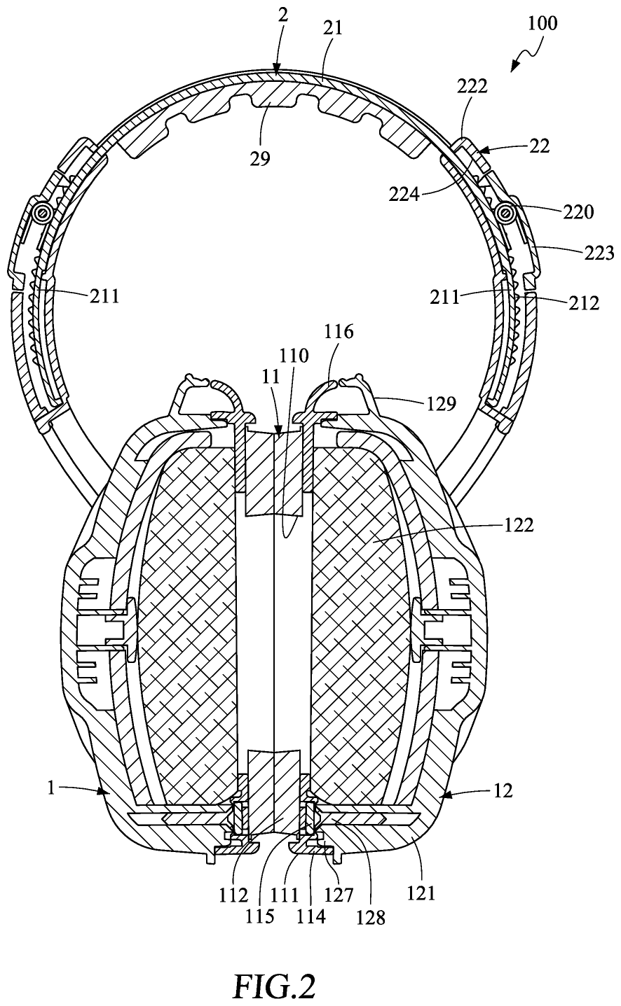

As shown in FIGS. 1 and 2, a head-mounted earmuff device 100 according to one embodiment of the present invention comprises: two earmuff bodies 1 and a headband body 2.

As shown in FIGS. 1 to 3, each earmuff body 1 has an earpad portion 11 and a housing portion 12. An inner side surface of the earpad portion 11 serves as a wearing surface which abuts a user's head, and the earpad portion 11 is provided with an accommodating hole 110 formed through the earpad portion 11 from the inner side surface of the earpad portion 11 to an outer side surface of the earpad portion 11. The housing portion 12 is pivotably connected to the earpad portion 11 in a manner that the housing portion 12 is pivoted with respect to the earpad portion 11 to switch between a closed position and an opened position.

Specifically, as shown in FIGS. 1 to 3, in the head-mounted earmuff device 100 according to the embodiment of the present invention, the earpad portion 11 of the earmuff body 1 includes an annular frame 111 and a cushion body 112 mounted to an inner side surface of the annular frame 111.

Furthermore, as shown in FIGS. 1 to 3, in the closed position, the housing portion 12 covers the outer side surface of the earpad portion 11 so that the accommodating hole 110 is closed to isolate environmental sounds. In the opened position, the housing portion 12 is pivoted to leave away from the outer side surface of the earpad portion 11 so that the accommodating hole 110 is opened to allow reception of the environmental sounds.

As shown in FIGS. 1 to 3, in the head-mounted earmuff device 100 according to the embodiment disclosed by the drawings of the present invention, the housing portion 12 of the earmuff body 1 has an outer case 121 and a soundproof block 122. The outer case 121 is pivotably connected to the annular frame 111, the soundproof block 122 is disposed in the outer case 121, and the soundproof block 122 is arranged to abut an outer side surface of the annular frame 111 in the closed position to close the accommodating hole 110 and leave away from the annular frame 111 in the opened position to open the accommodating hole 110.

In detail, as shown in FIGS. 1 to 3, in the head-mounted earmuff device 100 according to the embodiment of the present invention, the annular frame 111 is provided with a pivoting base 116 located in a top part of the annular frame 111, and the outer case 121 is provided with a pivoting portion 129 located in a top part of the outer case 121. The outer case 121 is pivotably connected to the annular frame 111 by connecting the pivoting portion 129 to the pivoting base 116. The outer case 121 is flipped to the opened position, taking the pivoting base 116 as a pivot center, from bottom to top with respect to the annular frame 111 by a side pulling force applied to the outer case 121 to leave away from the annular frame 111, and the outer case 121 is pivoted from the opening position to the closing position by gravity when the side pulling force is removed.

In addition, as shown in FIGS. 1 to 3, in the head-mounted earmuff device 100 according to the embodiment of the present invention, the annular frame 111 is provided with a magnetic member 115, the outer case 121 is provided with a corresponding magnetic element 128 corresponding to the magnetic member 115 so that the housing portion 12 and the earpad portion 11 are fixed in the closed position by a mutual magnetic attraction between the magnetic member 115 and the corresponding magnetic element 128.

Furthermore, as shown in FIGS. 1 to 3, in the head-mounted earmuff device 100 according to the embodiment of the present invention, the annular frame 111 is provided with an outer-seal flange 114 protruding outward from the annular frame 111, and the outer case 121 is provided with an inner-seal flange 127 protruding inward from the outer case 121. An outer-annular contour of the inner-seal flange 114 is corresponding in shape to an inner-annular contour of the outer-seal flange 127. In other words, in the closed position, the inner-seal flange 127 of the outer case 121 is air-tightly connected to the outer-seal flange 114 of the annular frame 111 to prevent the environmental sounds from entering through an aperture between the annular frame 111 and the outer case 121.

As shown in FIGS. 1 to 3, the headband body 2 has a mounting member 21 and two assembling portions 22. The mounting member 21 has two mounting sections 211 respectively extending in an arc toward left side and right side of the mounting member 21.

As shown in FIGS. 1 to 3, each assembling portion 22 has a pivoting end 221 extending downward and a sliding end 222 extending upward. The pivoting end 221 of the assembling portion 22 is pivotably connected to the earpad portion 11 of the earmuff body 1. The sliding end 222 of the assembling portion 22 is slidably connected to the mounting section 211 in an adjustable and fixable connecting length between the assembling portion 22 and the mounting member 21, and a dimension of a head-mounted space defined by the mounting member 21 and the two assembling portions 22 is adjustable by sliding and fixing the two assembling portions 22 with respect to the mounting member 21.

In detail, as shown in FIGS. 1, 2 and 5, in the head-mounted earmuff device 100 according to the embodiment of the present invention, a slide-fixing device is provided between the mounting section 211 of the mounting member 21 and the sliding end 222 of the assembling portion 22. The slide-fixing device includes a fixing rack 212 extending in the mounting section 211 along the mounting member 21 and a fixing detent 223 pivotably connected to the sliding end 222 of the assembling portion 22 in an elastically restorable manner. The fixing detent 223 has a fixing-protrusion part 22E which is detachably engaged with the fixing rack 212. A torsion spring 220 is disposed between the fixing detent 223 and the mounting member 21 to depress against the fixing detent 223 to maintain the fixing-protrusion part 22E of the fixing detent 223 to be engaged with the fixing rack 212. In other words, when an external pressing force is applied to the fixing detent 223 to overcome a biasing force of the torsion spring 220, the fixing detent 223 is upward pivoted so that the fixing-protrusion part 22E leaves away from the fixing rack 212, and thereby the assembling portion 22 is allowed to be slidable with respect to the mounting member 21.

Moreover, as shown in FIGS. 1, 2, 5 and 6, in the head-mounted earmuff device 100 according to the embodiment of the present invention, the sliding end 222 of the assembling portion 22 is provided with an accommodating recess 224 recessed inward from the sliding end 222 of the assembling portion 22 along the assembling portion 22 to form an arc accommodating space. The mounting section 211 of the mounting member 21 is mounted into the accommodating recess 224 along the assembling portion 22. A guide-protrusion part 213 protrudes from an arc inner side surface of the mounting member 21 along the mounting member 21. A guiding groove 225 is recessed inside the assembling portion 22 along the assembling portion 22. The guide-protrusion part 213 is mounted to the guiding groove 225 to guide a relative displacement between the mounting member 21 and the assembling portion 22.

Furthermore, as shown in FIGS. 1, 2 and 5, in the head-mounted earmuff device 100 according to the embodiment of the present invention, the mounting member 21 is provided with a stopping block 214 bent toward the mounting member 21 at an end part of the mounting section 211, and the assembling portion 22 is provided with a resisting block 226 located inside the assembling portion 22. When the mounting member 21 is outward extracted with respect to the assembling portion 22, the stopping block 214 knocks against the resisting block 226, and thereby the mounting member 21 and the assembling portion 22 are prevented from detaching each other.

In addition, as shown in FIGS. 1, 3 and 4, in the head-mounted earmuff device 100 according to the embodiment of the present invention, the annular frame 111 is provided with two pivoting studs 118 respectively protruding from a front side and a back side of the annular frame 111 so that the pivoting end 221 of the assembling portion 22 is pivotably connected to the two pivoting studs 118 protruding from the front side and the back side of the annular frame 111, and the pivoting stud 118 is provided with a pivot-stop block 117 outward protruding from the pivoting stud 118. The assembling portion 22 is provided at an inner side surface of the pivoting end 221 with an inner-restriction stop 229 and an outer-restriction stop 227 which are spaced apart from each other according to a pivot-restriction angle T. The pivot-stop block 117 is provided between the inner-restriction stop 229 and the outer-restriction stop 227 to confine the earmuff body 1 to pivot with respect to the assembling portion 22 in a pivot-angle range.

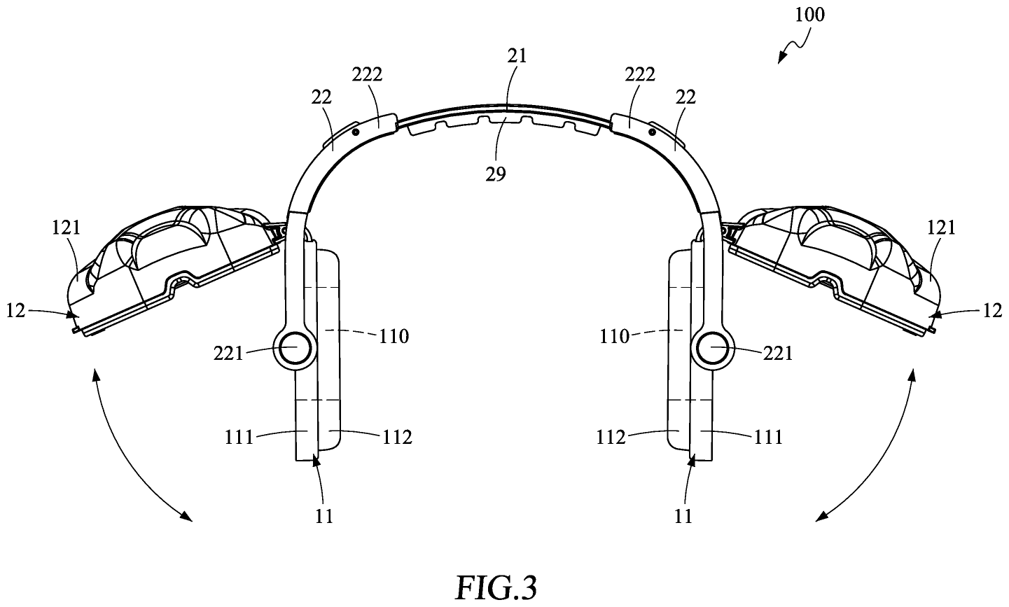

As shown in FIGS. 2 and 3, the headband body 2 further has an anti-sliding block 29. The anti-sliding block 29 is a long bar having multiple bumps, and the anti-sliding block 29 is disposed in the arc inner side surface of the mounting member 21. When the user wears the head-mounted earmuff device 100, the anti-sliding block 29 abuts the user's head so that the head-mounted earmuff device 100 is prevented from sliding on the user's head.

From the above, in the head-mounted earmuff device 100 of the present invention, the housing portion 12 is pivotably connected to the earpad portion 11 in a manner that the housing portion 12 is pivoted with respect to the earpad portion 11 to switch between the closed position and the opened position. Therefore, the housing portion 12 can cover the outer side surface of the earpad portion 11 to close the accommodating hole 110 to isolate the environmental sounds, and the housing portion 12 can be pivoted to leave away from the outer side surface of the earpad portion 11 to open the accommodating hole 110 to allow reception of the environmental sounds.

Moreover, in the head-mounted earmuff device 100 of the present invention, the dimension of a head-mounted space defined by the mounting member 21 and the two assembling portions 22 is adjustable by sliding and fixing the two assembling portions 22 with respect to the mounting member 21.

The above description should be considered as only the discussion of the preferred embodiments of the present invention. However, a person having ordinary skill in the art may make various modifications without deviating from the present invention. Those modifications still fall within the scope of the present invention.

What is claimed is:

1. A head-mounted earmuff device comprising:
two earmuff bodies, each earmuff body having an earpad portion and a housing portion, wherein an inner side surface of the earpad portion serves as a wearing surface which abuts a user's head, the earpad portion is provided with an accommodating hole formed through the earpad portion from the inner side surface of the earpad portion to an outer side surface of the earpad portion, and the housing portion is pivotably connected to the earpad portion in a manner that the housing portion is pivoted with respect to the earpad portion to switch between a closed position and an opened position, wherein in the closed position, the housing portion covers the outer side surface of the earpad portion so that the accommodating hole is closed to isolate environmental sounds, and in the opened position, the housing portion is pivoted to leave away from the outer side surface of the earpad portion so that the accommodating hole is opened to allow reception of the environmental sounds; and
a headband body having a mounting member and two assembling portions, wherein the mounting member has two mounting sections respectively extending in an arc toward left side and right side of the mounting member, the assembling portion has a pivoting end extending downward and a sliding end extending upward, the pivoting end of the assembling portion is pivotably connected to the earpad portion of the earmuff body, the sliding end of the assembling portion is slidably connected to the mounting section in an adjustable and fixable connecting length between the assembling portion and the mounting member, and a dimension of a head-mounted space defined by the mounting member and the two assembling portions is adjustable by sliding and fixing the two assembling portions with respect to the mounting member.

2. The head-mounted earmuff device as claimed in claim 1, wherein the earpad portion of the earmuff body includes an annular frame and a cushion body mounted to an inner side surface of the annular frame.

3. The head-mounted earmuff device as claimed in claim 2, wherein the annular frame is provided with two pivoting studs respectively protruding from a front side and a back side of the annular frame so that the pivoting end of the assembling portion is pivotably connected to the two pivoting studs protruding from the front side and the back side of the annular frame, and the pivoting stud is provided with a pivot-stop block outward protruding from the pivoting stud, the assembling portion is provided at an inner side surface of the pivoting end with an inner-restriction stop and an outer-restriction stop which are spaced apart from each other according to a pivot-restriction angle, the pivot-stop block is provided between the inner-restriction stop and the outer-restriction stop to confine the earmuff body to pivot with respect to the assembling portion in a pivot-angle range.

4. The head-mounted earmuff device as claimed in claim 2, wherein the housing portion of the earmuff body has an outer case and a soundproof block, the outer case is pivotably connected to the annular frame, the soundproof block is disposed in the outer case, and the soundproof block is arranged to abut an outer side surface of the annular frame in the closed position to close the accommodating hole and leave away from the annular frame in the opened position to open the accommodating hole.

5. The head-mounted earmuff device as claimed in claim 4, wherein the annular frame is provided with a pivoting base located in a top part of the annular frame, the outer case is provided with a pivoting portion located in a top part of the outer case, the outer case is pivotably connected to the annular frame by connecting the pivoting portion to the pivoting base, the outer case is flipped to the opened position, taking the pivoting base as a pivot center, from bottom to top with respect to the annular frame by a side pulling force applied to the outer case to leave away from the annular frame, and the outer case is pivoted from the opening position to the closing position by gravity when the side pulling force is removed.

6. The head-mounted earmuff device as claimed in claim 4, wherein the annular frame is provided with a magnetic member, the outer case is provided with a corresponding magnetic element corresponding to the magnetic member so that the housing portion and the earpad portion are fixed in the closed position by a mutual magnetic attraction between the magnetic member and the corresponding magnetic element.

7. The head-mounted earmuff device as claimed in claim 4, wherein the annular frame is provided with an outer-seal flange protruding outward from the annular frame, the outer case is provided with an inner-seal flange protruding inward from the outer case, an outer-annular contour of the inner-seal flange is corresponding in shape to an inner-annular contour of the outer-seal flange, wherein in the closed position, the inner-seal flange of the outer case is air-tightly connected to the outer-seal flange of the annular frame to prevent the environmental sounds from entering through an aperture between the annular frame and the outer case.

8. The head-mounted earmuff device as claimed in claim 1, wherein a slide-fixing device is provided between the mounting section of the mounting member and the sliding end of the assembling portion, the slide-fixing device includes a fixing rack extending in the mounting section along the mounting member and a fixing detent pivotably connected to the sliding end of the assembling portion in an elastically restorable manner, wherein the fixing detent has a fixing-protrusion part which is detachably engaged with the fixing rack, a torsion spring is disposed between the fixing detent and the mounting member to depress against the fixing detent to maintain the fixing-protrusion part of the fixing detent to be engaged with the fixing rack, and when an external pressing force is applied to the fixing detent to overcome a biasing force of the torsion spring, the fixing detent is upward pivoted so that the fixing-protrusion part leaves away from the fixing rack, and thereby the assembling portion is allowed to be slidable with respect to the mounting member.

9. The head-mounted earmuff device as claimed in claim 1, wherein the sliding end of the assembling portion is provided with an accommodating recess recessed inward from the sliding end of the assembling portion along the assembling portion to form an arc accommodating space, the mounting section of the mounting member is mounted into the accommodating recess along the assembling portion, a guide-protrusion part protrudes from an arc inner side surface of the mounting member along the mounting member, a guiding groove is recessed inside the assembling portion along the assembling portion, the guide-protrusion part is mounted to the guiding groove to guide a relative displacement between the mounting member and the assembling portion.

10. The head-mounted earmuff device as claimed in claim 9, wherein the mounting member is provided with a stopping block bent toward the mounting member at an end part of the mounting section, the assembling portion is provided with a resisting block located inside the assembling portion, when the mounting member is outward extracted with respect to the assembling portion, the stopping block knocks against the resisting block, and thereby the mounting member and the assembling portion are prevented from detaching each other.
